(19) United States
(12) Patent Application Publication
(10) Pub. No.: US 2012/0070476 A1
(43) Pub. Date: Mar. 22, 2012

(54) Compositions and Methods for Inactivation of Pathogens at Genital Tract Surfaces

(76) Inventors: Thomas R. Moench, Baltimore, MD (US); Deirdre E. O'Hanlon, Baltimore, MD (US); Richard A. Cone, Baltimore, MD (US)

(21) Appl. No.: 13/322,606

(22) PCT Filed: May 28, 2010
(86) PCT No.: PCT/US10/36589

§ 371 Date: Nov. 28, 2011

(30) Foreign Application Priority Data
May 29, 2009 (US) PCT/US2009/045706

(51) Int. Cl. A61K 9/00; A61P 31/00; A61P 31/04; A61M 5/00; A61P 31/18; A61P 33/02; A61K 31/366; A61M 25/00; A61K 31/19; A61P 31/22

(52) U.S. Cl. 424/400; 514/557; 514/452; 604/264; 604/140

(57) ABSTRACT

The present disclosure includes compositions and methods of inactivating pathogens of a genital tract of a female. The compositions include L-lactic acid substantially free of D-lactic acid. In particular, an intravaginal ring for sustained release of L-lactic acid out of a reservoir containing multi-gram quantities of L-lactic acid, with minimal osmotically induced swelling or pressurization of the reservoir during prolonged use is provided.

### PRIORITY CLAIM

This application is a continuation-in-part and claims the benefit of International Application No. PCT/US2009/045706 filed on May 29, 2009, which claims priority to U.S. Provisional Application No. 61/057,691, filed May 30, 2008.

### BACKGROUND

Genital tract infections are common and significant problems. They affect both men and women. Genital tract infections may be sexually transmitted as with genital herpes, human immunodeficiency virus (HIV/AIDS), gonorrhea, or chlamydia. Genital tract infections, particularly in the vagina, may also be caused by excessive growth of endogenous microbes such as yeast or anaerobic bacteria and other bacteria associated with conditions such as bacterial vaginosis (BV). Genital tract infections are known to cause uncomfortable symptoms, increase the risk of a variety of negative reproductive health outcomes, and carry the risk of transmission of infections to sexual partners.

Various intravaginal compositions exist that inhibit or kill pathogens and are intended as therapeutics or preventives. One class of compositions includes antibiotics, which are commonly used to treat genital tract infections. However, due to the risk of induction of resistance of pathogens to these agents, superinfection with new and more resistant pathogens after suppression of the original pathogens, or other antibiotic toxicities, repeated use of such compositions is disadvantageous.

The healthy human vagina has an acidic pH, which inhibits a variety of pathogens. A predominant source of maintaining this protective vaginal acidity is a bacterial flora dominated by lactobacillus species, which metabolize glycogen to glucose and glucose to lactic acid. The bacterial flora produce both the D- and the L-forms of lactic acid, and both forms have been shown to be present in the human vagina.

Measurements of lactic acid in the human vagina indicate that physiologic concentrations average about 1.2% lactic acid which acts as a buffering species to maintain a low vaginal pH. Acidic pharmaceutical compositions have been used to mimic this natural vaginal protective mechanism and to inactivate a variety of sexually transmitted pathogens, as well as potentially harmful endogenous vaginal flora. In particular, low molecular weight acid buffers have been used in therapeutic and preventative compositions at concentrations that are higher than those naturally found in the vagina. These higher buffer concentrations are required to extend the duration of action of the acid buffer, and for some applications, to overcome the strong tendency of the alkaline buffering capacity of semen to compromise the protective vaginal acidity after unprotected intercourse. However, a low pH alone is inadequate for acid inactivation of certain pathogens and to overcome the alkalinizing power of semen. Although acidity and lactic acid serve as a natural physiological vaginal protective mechanism, pharmacological compositions employing supra-physiologic concentrations of lactic acid or high concentrations of acid buffers, and particularly highly permeable buffers such as low molecular weight organic acids including racemic lactic acid (and equimolar mixture of D- and L-lactic acid), result in toxicity to cervicovaginal epithelia due to acid permeation into cells and/or hypertonicity.

Women who are susceptible to BV often experience frequent relapses and remissions of the condition. The literature shows high rates of recurrence of BV even after successful treatment. Thus, providing a therapeutic composition episodically is unlikely to be successful over the long term. In addition, delivery of an adequate dose without a sustained-release device, such as with a gel, may be limited by its duration of action due to a more rapid loss of such dosing forms along with the natural shedding of vaginal fluids.

In order to achieve desirable therapeutic levels of lactic acid for sustained suppression of BV with lactic acid, however, the amount of material required is large relative to the amount typically released from sustained-release vaginal rings. Sustained-release vaginal rings are typically loaded with between a few mg and a few hundred mg of drug. For example, devices designed to release lactic acid from bioerodable polymers are limited in the amount of lactic acid that can be loaded and released from the device and in the rate of its release. Moreover, the release of other co-monomers may have unforeseen epithelial toxicities, or may deleteriously alter the vaginal flora by inhibitory mechanisms or by serving as metabolic substrates and stimulating the growth of harmful organisms. In order to effectively inhibit BV by mimicking the natural supply of lactic acid, much higher lactic acid loading and rates of delivery are required, since, when present, vaginal lactobacilli continuously supply lactic acid at a high rate to keep up with its constant loss by diffusion through, and metabolism by, the cervicovaginal epithelium.

The requirement for loading a relatively large quantity of lactic acid in a sustained-release device to enable extended delivery of a therapeutic amount of lactic acid produces a significant osmotic load within the device creating a tendency for the sustained-release device to draw water into the device and causing it to swell. Swelling of the sustained-release device may lead to undesirable changes in the dimensions and stiffness of the device in addition to potentially inducing leakage of now pressurized internal fluid.

There is, therefore, a need for improved compositions and methods to prevent and/or treat genital tract infections, and, in particular, there is a need for sustained release delivery of a non-antibiotic therapeutic and preventative compositions with improved safety profiles suitable for treatment of frequently recurring conditions or for repeated or sustained applications to prevent infections.

### SUMMARY

The present disclosure is related to compositions having potent activity to inactivate pathogens at the epithelial surfaces such as the genital tract, while minimizing epithelial toxicity. The compositions comprise L-lactic acid substantially free of D-lactic acid. It has been surprisingly found that the use of the L-stereoisomer of lactic acid reduces the potential for genital tract epithelial toxicity compared to the potential that exists due to the presence of D-lactic acid or the racemic mixture containing equal parts of D- and L-lactic acid. Moreover, it has been surprisingly found that significant pathogens, including the herpes simplex viruses that cause genital tract infections, are inactivated more efficiently by L-lactic acid than by D-lactic acid.

In addition we have found lactic acid to be a highly effective microbicide with activity against important sexually transmitted pathogens as well as endogenous pathogens and to provide enhanced activity against many pathogens compared to that achieved at the same pH in the absence of lactic acid. Furthermore, we have found that although racemic lactic acid is a potent microbicide, L-lactic acid substantially free of D-lactic acid has a superior therapeutic index when compared to D-lactic acid or the racemic mixture of D- and L-lactic acid.

We have also found that lactic acid can be provided in relatively high amounts in a well-retained and rapidly and continuously releasing form, such as a vaginal ring, while avoiding the problem of excessive osmotic swelling of the device when exposed to vaginal fluids. In addition, we have found that loading the device with lactic acid in solid form reduces an otherwise problematic swelling of the ring caused by the osmotic activity of this quantity of lactic acid. Furthermore, we have found that the use of polyurethane as the diffusion barrier permits adequate diffusion of the lactic acid out of the device while limiting ring swelling due to diffusion of water into the device.

Accordingly, the present disclosure provides a pharmaceutical composition comprising a therapeutically effective amount of L-lactic acid, substantially free of D-lactic acid, for administration to the vagina of a human. The present disclosure also provides a sustained-release device capable of delivering large quantities of lactic acid while avoiding problems associated with osmotic swelling.

In an embodiment, the therapeutically effective amount of L-lactic acid is sufficient to maintain an L-lactic acid concentration of 0.2% to 2.0% at the epithelial surface of the vagina.

In an embodiment, the composition includes at least one additional buffering agent.

In an embodiment, the additional buffering agent has a molecular weight of at least 25,000.

In an embodiment, the additional buffering agent is selected from the group consisting of cross-linked carboxylic acids, polyacrylic acids, crosslinked polyacrylic acids, carbomers, polycarbophils, carboxylated polysaccharides, carboxycellulose, carboxymethylcellulose, alginic acid and combinations thereof.

In an embodiment, the composition is a sustained-release composition.

In an embodiment, the sustained-release composition includes particles formed from a polymer comprised of L-lactic acid monomers or L-lactide monomers. The particles have a size of between about 10 nanometers and about 10,000 nanometers.

In an embodiment, the polymer includes a co-monomer of glycolic acid.

In an embodiment, the sustained-release composition includes at least one sustained-release component adapted to administer the therapeutically effective amount of L-lactic acid to an epithelial surface within the vagina at a rate of about 1 milligram to about 1000 milligrams per day.

In an embodiment, the sustained-release component is selected from the group consisting of a polymer, an L-lactic acid-permeable membrane, an L-lactic acid-impregnated matrix, an L-lactic acid-permeable matrix, a sponge, a bioerodable material, and combinations thereof.

In an embodiment, the membrane defines a reservoir portion that contains L-lactic acid.

Another embodiment of the present disclosure provides a method for reducing pathogenic infection at a vaginal epithelial surface comprising administering to said epithelial surface a composition comprising a therapeutically effective amount of L-lactic acid substantially free of D-lactic acid.

In an embodiment, the pathogenic infection is selected from the group consisting of bacterial vaginosis, Chlamydia trachomatis, Neisseria gonorrhoeae, herpes simplex virus type 1, herpes simplex virus type 2, human immunodeficiency virus (HIV), Trichomonas vaginalis, and any combination thereof.

In an embodiment, the composition includes a sustained-release component adapted to release the L-lactic acid at a rate of between about 1 and about 1000 milligrams per day.

In an embodiment, the rate of release of L-lactic acid provides a concentration of 0.2 to 2% at the epithelial surface.

A further embodiment provides a method of maintaining pH at an epithelial surface comprising administering to said epithelial surface a composition comprising a therapeutically effective amount of L-lactic acid substantially free of D-lactic acid.

In an embodiment, the pH at the epithelial surface is maintained between about 3.1 and about 4.2.

An additional embodiment provides an intravaginal composition comprising a tubular ring having an inner surface and an outer surface. The outer surface of the tubular ring is configured to contact the epithelial surface of the vagina. The intravaginal composition also comprises a membrane portion of the tubular ring continuous with a space defined by the inner surface of the tubular ring, and an L-lactic acid composition, substantially free of D-lactic acid. The L-lactic acid composition is contained within the space and the L-lactic acid composition is capable of diffusing through the membrane portion of the tubular ring to contact the epithelial surface of the vagina.

In an embodiment, the tubular ring comprises silicone.

In an embodiment, the tubular ring includes an outer diameter between about 50 mm and about 100 mm.

In an embodiment, the tubular ring includes a cross-sectional diameter between about 4 mm and about 15 mm.

In an embodiment, the amount of L-lactic acid composition contained within the tubular ring space is between about 2 grams and about 5 grams of the composition.

In an embodiment, the L-lactic acid composition is in the form of a powder.

In an embodiment, the L-lactic acid composition includes less than 5% D-lactic acid.

In an embodiment, the composition is substantially free of D-lactic acid, and the composition has reduced toxicity to an epithelial surface of a human.

In an embodiment, the epithelial surface is associated with a genital tract.

In an embodiment, the genital tract is female.

In an embodiment, composition is a semi-solid form.

Another embodiment of the present disclosure includes a method for inhibiting pathogenic infection at an epithelial surface of a mammal, either to prevent a new infection, or to treat an established infection. The method comprises providing a pharmaceutical composition for administration to the epithelial surface. The composition provides a concentration of between about 0.2 and about 2% L-lactic acid to the epithelial surfaces. The composition is substantially free of D-lactic acid, and the composition is non-toxic to an epithelial surface of a human.

An additional embodiment of the present disclosure includes a composition comprising at least one polymer including a plurality of monomers. At least one of the monomers is L-lactide substantially free of D-lactide. The polymer is adapted to release monomeric L-lactic acid.

In an embodiment, the composition is provided in a solid dosage form.

In an embodiment, the solid dosage form includes a polymer in the form of a particle having a size of between about 10 nanometers and about 10,000 nanometers.

In an embodiment, the composition includes a co-monomer.

Yet a further embodiment of the present disclosure includes a composition configured to release L-lactic acid, substantially free of D-lactic acid, at a rate. In an embodiment the release rate is between 1 milligram per day and 1000 milligrams per day.

In an embodiment, the composition includes a structure permeable to L-lactic acid.

In an embodiment, the structure is a matrix containing L-lactic acid in a polymer permeable to the diffusion of L-lactic acid.

In an embodiment, the L-lactic acid is contained in a reservoir space within a structure comprised of a polymer permeable to the diffusion of L-lactic acid.

Another embodiment of the present disclosure includes the use of L-lactic acid in the manufacture of a medicament or prevention for the treatment of a viral, bacterial, fungal, or protozoal infection of a genital tract of a female.

A further embodiment of the present disclosure includes a device comprising at least one wall separating a composition from a genital tract surface. The composition includes at least one gram of lactic acid. The permeability of the wall for the lactic acid is at least half the permeability of the wall for water.

In an embodiment, the wall is in the form of a tube.

In an embodiment, the tube is in the shape of a ring.

In an embodiment, the tubular ring includes an outer diameter of between about 50 nun and about 100 mm and a cross-sectional diameter between about 4 mm and about 15 mm.

In an embodiment, the wall includes a silicone polymer.

In an embodiment, the lactic acid is in a powder form.

In an embodiment, the wall includes a polyurethane polymer.

In an embodiment, the lactic acid is in a liquid or powder form.

In an embodiment, the composition includes at least one form of lactic acid selected from the group consisting of L-lactic acid, calcium lactate and L,L-lactide.

In an embodiment, the composition includes between about 2 grams and about 8 grams of lactic acid.

In an embodiment, the lactic acid passes through the wall at a rate of about 1 milligram to about 1000 milligrams per day.

An additional embodiment of the present disclosure includes a method of decreasing the pH of a genital tract. The method includes positioning within the genital tract for at least one week a device comprising at least one wall separating a composition from the genital tract surface. The composition comprises at least one gram of lactic acid. The permeability of the wall for the lactic acid is at least half the permeability of the wall for water.

In an embodiment, the pH of the genital tract is decreased to between about 3.1 and about 4.2.

Another embodiment of the present disclosure includes a method of treating a pathogenic infection of a genital tract. The method includes positioning for at least one week within the genital tract a device comprising at least one wall separating a composition from the genital tract surface. The composition comprising at least one gram of lactic acid. The permeability of the wall for the lactic acid is at least half the permeability of the wall for water. The pathogenic infection is selected from the group consisting of bacterial vaginosis, Chlamydia trachomatis, Neisseria gonorrhoeae, herpes simplex virus type 1, herpes simplex virus type 2, human immunodeficiency virus (HIV), Trichomonas vaginalis, and combinations thereof.

Another embodiment of the present disclosure includes a device for delivering a high dose of lactic acid to a genital tract. The lactic acid is in a substantially powder form. The device also includes a wall separating the lactic acid from a surface of the genital tract. The wall includes a polyurethane polymer. The permeability of the wall for the lactic acid is at least half the permeability of the wall for water. The device further includes a compartment including a gas, wherein the compartment extends along the length of the device.

In an embodiment, compression is applied to the device during loading the device with the lactic acid, and the compression is released after loading the device with the lactic acid.

A further embodiment of the present disclosure includes a genital tract surface device. The device includes a tubular ring having a wall defining a space within the tubular ring. The space contains at least one gram of an L-lactic acid composition, substantially free of D-lactic acid. The rate of diffusion of L-lactic acid from the space through the wall of the tubular ring is at least half the rate of diffusion of water through the wall into the space.

In an embodiment, the wall includes a polyurethane polymer.

In an embodiment, the lactic acid is in a substantially powder form.

In an embodiment, the rate of diffusion of L-lactic acid from the space through the wall of said tubular ring is at least twice the rate of diffusion of water through the wall into the space.

It is therefore an advantage of the compositions and methods of the present disclosure to prevent vaginal infection with very low epithelial toxicity and very low toxicity to the normal vaginal flora.

Yet another advantage of the compositions and methods of the present disclosure includes providing treatment of established vaginal infections with less toxicity than other compositions and methods such as antibiotic treatments.

A further advantage of the compositions and methods of the present disclosure includes preventing transmission of sexually transmitted pathogens between women and men by inactivating pathogens in the vagina.

An additional advantage of the compositions and methods of the present disclosure includes actively supporting the dominance of the normal vaginal lactobacillus flora in opposition to harmful flora.

An additional advantage of the compositions and methods of the present disclosure includes reducing the potential for epithelial damage from high concentrations of permeant acids while providing the highest possible efficacy with minimal toxicity to the user, i.e., maximum therapeutic or prophylactic index.

An additional advantage of the composition and methods of the present disclosure includes providing sustained levels of the protective or therapeutic concentrations of lactic acid.

Additional features and advantages of the present invention are described in and will be apparent from the following Detailed Description.

### BRIEF DESCRIPTION OF THE FIGURES

FIG. 1 is a graph showing the inactivation of Herpes Simplex Virus Type 2 (HSV-2) after acidification with or without L-lactic acid or D-lactic acid.

FIG. 2 is a graph showing the concentration of lactic acid and pH in human vaginal secretions.

FIG. 3 is a graph comparing the toxicity of D- and L-lactic acid after application to epithelial cell monolayers in tissue culture.

FIG. 4 is a graph showing the potency of racemic lactic acid in killing bacterial vaginosis-associated bacteria and not harming lactobacilli.

FIG. 5 is a graph showing the release of L-lactic acid from sustained-release compositions.

FIG. 6A is a cross sectional view of one side of an intravaginal ring illustrating a free bubble of gas in the lumen of the device, with the gas making extensive contact with the inner wall.

FIG. 6B is a cross sectional view of one side of an intravaginal ring illustrating a single cylindrical gas compartment floating upward in the lumen of the device and making tangential content with the inner wall.

FIG. 6C is a cross sectional view of one side of an intravaginal ring illustrating a multiple spherical gas compartment floating upward in the lumen of the device and making tangential content with the inner wall.

### DETAILED DESCRIPTION

The present disclosure relates to compositions and methods for the prevention or treatment of microbial pathogens. In particular, the present disclosure relates to compositions including the L-stereoisomer of lactic acid, substantially free of the D-stereoisomer of lactic acid, and methods of using such compositions. The present disclosure further relates to acid-buffering compositions that include the L-stereoisomer of lactic acid, substantially free of the D-stereoisomer, as an active agent to improve the therapeutic and prophylactic indices of such acid-buffering compositions.

Lactic acid exists as stereoisomers or enantiomers designated D-lactic acid (also referred to as (−)-lactic acid and (R)-lactic acid), and L-lactic acid (also referred to as (+)-lactic acid and (S)-lactic acid). The two stereoisomers have the same chemical formula, but are two distinct molecules that are mirror images of each other. The middle carbon of the three carbon backbone is a tetrahedrally-bonded carbon with four different substituents bonded to it (a carboxyl group, a methyl group, a hydroxyl group, and a hydrogen atom), resulting in two possible chiral or mirror image forms depending on the placement of these four substituents.

It has been shown that vaginal secretions contain both D- and L-lactic acid (Boskey, 2001). Thus, a composition containing the racemic mixture of D- and L-lactic acid stereoisomers would appear to be an appropriate physiological composition for use within the intravaginal space.

It has been surprisingly found, however, that the L-stereoisomer of lactic acid is less toxic to the vagina than the D stereoisomer of lactic acid and is a more potent inactivator of sexually transmitted pathogens. It has also been determined that quantities of L-lactic acid, substantially free of D-lactic acid, improve compositions including acidic buffering compositions for the prevention and treatment of pathogenic infection of epithelial surfaces.

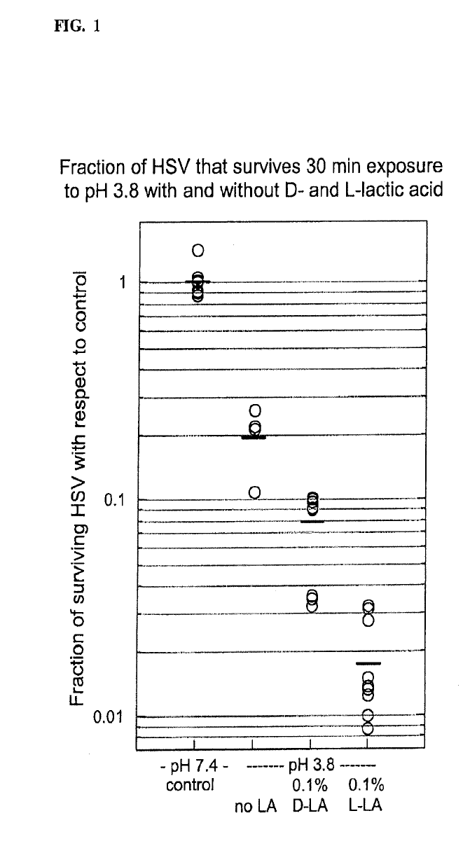

FIG. 1 illustrates the inactivation of HSV-2 after acidification with or without L-lactic acid or D-lactic acid. A pH of 3.8 without lactic acid reduced HSV-2 viability approximately 5-fold. Both D- and L-lactic acid further reduced HSV-2 viability. L-lactic acid, however, reduced HSV-2 viability approximately 11-fold more than low pH without lactic acid, and approximately 5-fold more than low pH with D-lactic acid. In contrast, acetic acid did not increase the degree of inactivation beyond the level caused by low pH without organic acids.

Without being bound to any particular theory, we have demonstrated a mechanism of inactivation by organic acids that is different from the conventional “small anion trapping” mechanism thought to explain the inactivation of pathogens. It has long been believed that small, permeable, organic acids such as acetic acid and lactic acid increase killing of many pathogens by the mechanism of “small anion trapping”.

According to the “small anion trapping” theory, small, membrane permeable, organic acids enhance pathogen killing by permeating the pathogen cell membrane and by accumulating in the cytoplasmic space. Initially, the extracellular space has a low pH due to the presence of the small acid. At this pH, the acid is non-dissociated and uncharged making the small organic acid membrane-permeant. The acid crosses the membrane and arrives in the cytoplasm. Here the pH is initially high, and the acid releases its ionizable hydrogen ion, and thereby also creates the charged anionic form of the acid. This form cannot re-cross the membrane due to its negative charge, and so accumulates inside the cell. Thus, according to this theory, small weak acids provide a mechanism for efficient transfer of hydrogen ions to the interior of the pathogen, resulting in acidification and high and potentially toxic concentrations of the acid's anion within the pathogen.

Considering this prevalent theory of small weak acid inactivation of pathogens, it is not surprising that attention has not been given to the potentially different activities of the two distinct stereoisomers of lactic acid, since both are identical in their buffering character (both have the same pKa), and are identical in their size and degree of hydrophobicity. Thus, they would be expected to perform equally well as permeant acid buffers, with equal access to, and trapping within, the pathogen interior. This is particularly true in the case of viral pathogens, which have no energy driven transport mechanisms (“pumps”) to export toxic molecules accumulating in their interior, and moreover, have no means of maintaining an internal pH different from the pH of the fluid they are suspended in. Thus the knowledge in the art would not suggest the preferential use or avoidance of one or the other (D- or L-) stereoisomer of lactic acid for inactivation of pathogens.

The data illustrated in FIG. 1, however, suggest that viruses are not inactivated by this small anion trapping mechanism. First, the small anion trapping mechanism is implausible for viral pathogens, since viruses have no metabolic activity, and hence cannot maintain a pH gradient between the viral interior and exterior, a condition necessary for this mechanism to work. Most importantly, the stereo specificity of the effect (the substantially greater effect of L-lactic acid over D-lactic acid), in an environment where there cannot be stereo specific organic acid export pumps (a non-metabolically active virus), indicates a mechanism other than small anion trapping. Therefore, one would not have expected the differential effect of L-lactic acid vs. D-lactic acid on the inactivation of viral pathogens.

In an embodiment of the present disclosure, a therapeutically effective amount of L-lactic acid, substantially free of D-lactic acid, is administered to an epithelial surface of a genital tract. As referred to herein, a therapeutically effective amount of L-lactic acid refers to a an amount of L-lactic acid delivered to the epithelial surface of a genital tract capable of producing a desired physiological effect such as maintaining the pH, providing a microbicidal concentration of L-lactic acid, and preventing or reducing pathogenic infection. As referred to herein, substantially free of D-lactic acid refers to a concentration of D-lactic acid in the composition that does not exceed more than 20% of the total lactic acid concentration, or more preferably, does not exceed more than 5% of the total lactic acid concentration, or even more preferably, does not exceed more than 1% of the total lactic acid concentration. As referred to herein, genital tract refers to any portion of the structures from the ovaries to the vulva in a female or from the testicles to the external urethral meatus in a male. As referred to herein, epithelial cells refer to any cells that line the inside cavities and lumen of the body of a subject including the genital tract.

L-lactic acid may be produced commercially by chemical synthesis, and more economically, by fermentation from various carbohydrate feedstock substrates including glucose, sucrose, maltose, other mono and disaccharides, or starches, depending on the fermenting organism to be used. Organisms useful for this purpose include fungi, such as Rhizopus oryzae and others, lactic-acid-producing bacteria, such as various lactobacilli species, or other bacterial species. Organisms may be selected to produce predominately the L-lactic acid sterioisomer, followed if needed by further purification to isolate L-lactic acid substantially free of D-lactic acid. Alternatively, Lactobacillus helveticus or other organisms may be genetically modified to inactivate the D-lactate dehydrogenase gene, thus preventing the organism from synthesizing D-lactic acid. Such organisms are capable of high yield production of L-lactic acid with no or minimal production of D-lactic acid.

In an embodiment, a composition comprising a therapeutically effective amount of L-lactic acid, substantially free of D-lactic acid, is administered to an epithelial surface of a genital tract. The composition may include a concentration of L-lactic acid of between 0.2 and 2%, preferably between about 0.3% and about 1.8% or, more preferably, between about 0.8% and about 1.6%.

In an embodiment, the concentration of L-lactic acid, substantially free of D-lactic acid, in the composition is sufficient to produce a concentration of L-lactic acid of between 0.2 and 2%, preferably between about 0.3% and about 1.8% or, more preferably, between about 0.8% and about 1.6% at the epithelial surface.

In an embodiment of the present disclosure, the composition includes at least one high molecular weight buffering polymer in addition to L-lactic acid. Employing a high molecular weight buffering polymer avoids the need to employ supraphysiological concentrations of L-lactic acid to achieve adequate pH buffering capacity, and thus prevents excessive permeation of the L-lactic acid into the cells, hypertonicity and consequent toxicity. As referred to herein, toxicity can include any change in the epithelial cells that can increase susceptibility to infection. Such changes may further involve induction of inflammatory responses or killing of surface epithelial cells. Unlike low molecular weight buffering agents, high molecular weight polyvalent buffers cannot cross cell membranes and enter cells. Thus, compositions containing both L-lactic acid and an additional high molecular weight buffer achieve high total buffer capacity without using a toxic concentration of L-lactic acid. The addition of high molecular weight buffers may also be particularly helpful in products used for protection during or after sexual intercourse where semen exposure can neutralize all but very potent acidic buffering compositions.

In an embodiment, L-lactic acid, substantially free of D-lactic acid, is combined with at least one high molecular weight buffering polymer in a composition. The L-lactic acid may be present in an amount of between about 0.2% and about 2%, or more preferably between about 0.5% and about 1.8% and still more preferably between about 0.8% and about 1.6% w/w of the composition. The buffering polymer may have any suitable molecular weight such as a molecular weight greater than about 25,000, more preferably greater than about 100,000, still more preferably greater than about 1,000,000. The buffering polymer may include any suitable carboxylated polymer such as polyacrylic acids, crosslinked polyacrylic acids, carbomers, polycarbophils and combinations thereof. In an embodiment, the buffering polymer may include carboxylated polysaccharides including, without limitation, carboxycellulose, carboxymethylcellulose, alginic acid and combinations thereof.

In an embodiment, the L-lactic acid is provided in the form of a non-toxic polymer of L-lactide (a dimer of two L-lactic acid molecules), referred to herein as a poly-L-lactide or poly-L-lactic acid. In an embodiment, the described compositions comprise polymers that contain poly-L-lactic acid substantially free of D-lactic acid. In an embodiment, poly-L-lactide is included in compositions as poly-L-lactide particles where these particles are substantially free of D-lactic acid.

The poly-L-lactide particles may have a high surface-to-volume ratio with the size of the particles in an embodiment in the range of about 1 to about 10,000 nanometers in largest dimension. In another embodiment the poly-L-lactide particles are employed in diameters of 10 to 1,000 nanometers. Thus, the polymeric L-lactic acid may be in the form of nanospheres or microspheres capable of delivering large amounts of L-lactic acid without increasing the concentration of monomeric L-lactic acid beyond levels that would be toxic to the epithelial tissues. The lower concentration of monomeric L-lactic acid also reduces the loss of L-lactic acid across the epithelium that would otherwise be driven at a high rate by high concentrations of exclusively monomeric L-lactic acid. In an embodiment, poly-L-lactide particles are stored in a non-aqueous environment before use in order to prevent premature release of monomeric L-lactic acid by hydrolysis. In an embodiment, aqueous formulations are prepared shortly before use by having the end-user mix particles into an aqueous phase to avoid any instability during prolonged storage of aqueous formulations such as gels or other water containing formats. In other embodiments, poly-L-lactide particles may be incorporated into films, tablets, and other solid dosage forms that disintegrate when moistened in situ by vaginal fluids, or added to non-aqueous semi-solid vaginal dosage forms such as PEG-based or other meltable wax-like dosage forms.

In an embodiment, the poly-L-lactide particles of the described composition may comprise polymers that contain poly-L-lactide along with one or more other co-monomers, but substantially free of D-lactic acid. The nature and the proportions of such co-monomers may be chosen to adjust the release-rate of L-lactic acid. For example, combining the L-lactide monomer with a co-monomer of glycolic acid, poly-lactic-co-glycolic acid (PLGA) can be produced, and the hydrolysis rate altered by varying the ratio of the two co-monomers, with higher concentrations of glycolic acid increasing the hydrolysis rate.

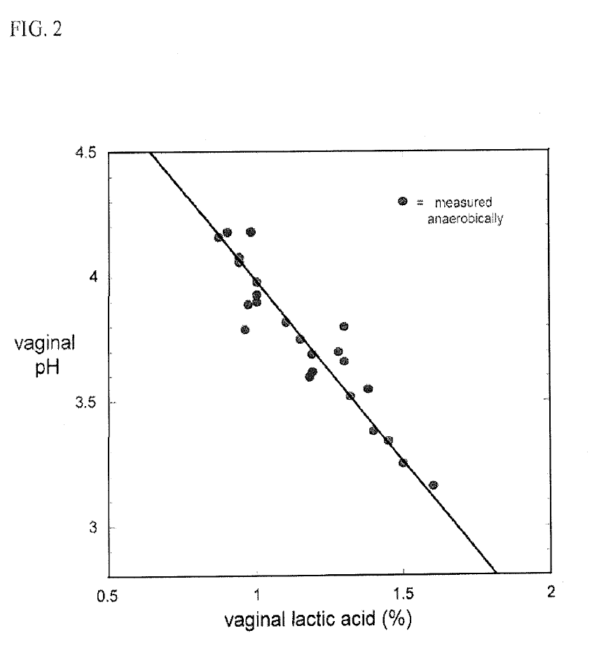

In an embodiment L-lactic acid is provided in a composition capable of sustained release over an interval of time, wherein the composition releases L-lactic acid at a rate sufficient to provide a concentration at the epithelial surface of between about 0.2 and about 2%, preferably between about 0.3% and about 1.8% or, more preferably, between about 0.8% and about 1.6%. The L-lactic acid may be administered or released from the sustained-release composition at a rate that will maintain the concentration of L-lactic acid at or near the physiological levels documented in FIG. 2. For example, as illustrated in FIG. 2, physiological levels of L-lactic acid may include a concentration of between 0.8 and 1.6% lactic acid. It should be appreciated that the composition may contain L-lactic acid at a concentration greater than the desired concentration to be released to the epithelial surface.

In an embodiment, the composition is administered topically to the epithelial surface of the genital tract of the subject. In an embodiment, L-lactic acid may be administered to the epithelial surface through sustained release strategies, wherein the L-lactic acid is released over time at a regulated rate to provide the ingredients at controlled rates and/or concentrations, and/or to extend the duration of action of the L-lactic acid. For example, L-lactic acid may be released from sustained-release compositions, including without limitation, vaginal rings, cervical barriers, sponges, and other intravaginally retained compositions. In an embodiment, the concentration of L-lactic acid, substantially free of D-lactic acid, in the sustained-release composition is sufficient to produce a concentration of L-lactic acid of between 0.2 and 2%, preferably between about 0.5% and about 1.8% or, more preferably, between about 0.8% and about 1.6% at the epithelial surface.

In an embodiment sustained release over an interval of time is achieved by storage of L-lactic acid within a space surrounded by a permeable material that allows release of L-lactic acid at a rate sufficient to achieve a concentration of L-lactic acid at an epithelial surface of between about 0.2 and about 2%, preferably between about 0.3% and about 1.8% or, more preferably, between about 0.8% and about 1.6%.

It should be appreciated that the release rate from this and other sustained-release compositions can be regulated by means known in the art, such as, without limitation, include varying the concentration of the L-lactic acid contained within the composition, the thickness and/or permeability of the surrounding permeable material, the total surface area of the composition, and the pH of the lactic acid in the composition, the latter adjusted by various degrees of partial neutralization with various alkaline materials such as sodium hydroxide calcium carbonate, and the like.

It should also be appreciated that other materials may be employed in the disclosed compositions. The materials may be chosen for appropriate permeability of lactic acid, and other types of sustained-release compositions may be substituted for those described herein, including, without limitation, matrix-type compositions where the L-lactic acid is disbursed within the permeable material of the composition, osmotically driven pump compositions, and bioerodable compositions.

In an embodiment, L-lactic acid, substantially free of D-lactic acid, is administered to an epithelial surface as a by-product of hydrolytic biodegradation of the poly-L-lactide into monomeric L-lactic acid. Polymers may be synthesized incorporating L,L-lactide (also known as L-lactide) as the sole monomer (creating poly-L-lactide), or as one of two or more co-monomers. For example, in an embodiment, a glycolic acid co-monomer is included in a composition to alter the physical properties of the copolymer, such as increasing its dissolution rate. Such polymers and copolymers may be included in a composition to release L-lactic acid by hydrolysis when placed in an aqueous environment. The release rate of L-lactic acid may be adjusted by varying the choice of, and proportion of, monomers and co-monomers. The L-lactic acid release rate may also be influenced by the surface to volume ratio of the particles. For example, release rate may be increased by forming the polymer into small particles, in the range of about 10 nanometers to about 10 microns.

The compositions may include other active pharmaceutical agents or excipients that may or may not affect the rate of release of the lactic acid from the composition. In an embodiment of the presently disclosed compositions, the composition includes at least one additional agent including, without limitation, preservatives other than L-lactic acid (such as methylparaben, propylparaben, benzoic acid, sorbic acid, disodium or other forms of ethylenediaminetetraacetic acid (EDTA)), tonicity- or viscosity-adjusting agents (such as glycerin, propylene glycol, polyethylene glycol), physiological salts (such as sodium chloride, potassium chloride, monobasic potassium or sodium phosphate, dibasic potassium or sodium phosphate), or additional active agents that inhibit, kill, or block harmful microorganisms. It should be appreciated that the concentration of L-lactic acid may be altered and that different quantities of carbomer, or other high molecular weight buffers (such as polycarbophil, or carboxylated polysaccharides such as alginic acid or carboxymethylcellulose) may be substituted for those described. It should be further appreciated that any suitable variation may be made in the concentrations of the ingredients of the described compositions to adjust osmotic strength, pH, viscosity, and yield strength.

The compositions of the present disclosure may be delivered in any suitable form such as non-solid, semi-solid or solid forms. Examples of non-solid dosage forms include, without limitation, gels, creams, ointments, or foams. An example of a semi-solid form includes, without limitation, a suppository. Examples of solid dosage forms include, without limitation, films, tablets, micro- or nanoparticles, or liquid-filled capsules.

In various sustained-release composition embodiments, L-lactic acid may be stored in solution, impregnated throughout an erodible coating or matrix, or contained in an inner reservoir portion that may include, for example, an initially drug-free membrane or “skin” permeant to L-lactic acid. In various embodiments, L-lactic acid may be released from such compositions by passing through the walls of the composition by diffusion through the material of the wall, or via diffusion or osmotic extrusion through pores, as with sponges or other porous materials. L-lactic acid may alternatively be extruded via osmotically activated pumps, or by erosion of a surrounding matrix, or by any other suitable release mechanism.

In an embodiment, L-lactic acid substantially free of D-lactic acid is released from a sustained-release device. Sustained-release devices may include an intravaginal ring, a cervical barrier device with a ring-shaped rim, vaginal sponges in ring, disk, or roughly spherical form, or any other suitable compositions and designs.

The dimensions of the sustained-release devices may be in the range of about 50 mm to about 100 mm, or between about 60 and about 80 mm or sized to be retained in the vagina. If configured as a ring, the overall diameter of the ring may be as described, and the cross-sectional diameter of the ring may be between about 4 and about 15 mm, between about 5 and about 12 mm, or between about 6 and about 9 mm. The thickness of the wall may be varied between about 0.5 and about 3 millimeters, or between about 1 and about 2 millimeters to adjust the stiffness of the device and to increase or decrease control of the rate of lactic acid release. Combinations of adequate stiffness and high release rates can be achieved by provision of reinforcing mesh or other structures closely applied to the inner surface of the wall, or elsewhere in the reservoir.

In an embodiment, lactic acid is delivered to an epithelial or genital tract surface through a wall of a sustained-release device. The wall of the device is permeable to the lactic acid composition allowing the lactic acid composition to pass through or diffuse through the wall and contact the epithelial surface. The wall of the sustained-release device may be permeable to water, but, in an embodiment, the permeability of the wall to water is less than about half that of the permeability of the wall to lactic acid. Permeability of the wall of a sustained-release device may be referred to as a ratio, on a mass basis, of the rate at which lactic acid exits the lumen of the device and the rate at which water enters the lumen of the device. In an embodiment, the wall of the sustained-release device provides a ratio of lactic acid exit rate to water entry rate of at least about 0.6 by weight. In another embodiment, the wall provides a ratio of lactic acid exit rate to water entry rate of at least about 1.0 by weight.

The wall of the sustained-release device may include any suitable material that allows at least half the rate of the enclosed lactic acid exit as the rate of water entry. For example, the wall may include polymers such as silicone, or any suitable polymerized siloxane, and polyurethane polymers, such as polyether and polyester polyurethanes or combinations thereof. Polyether polyurethanes, in particular, are well-suited to resist hydrolysis. Aromatic and aliphatic polyurethanes may be used. Aromatic polyurethanes, in particular, are known to soften less with exposure to moisture. In addition, the hydrophilicity of polyurethane can be varied in order to achieve different release rates, and less hydrophilic (such as Tecophilic® HP-60D-20, Lubrizol, Inc.) and more hydrophilic polyurethanes (such as Tecoflex® EG-85A, Lubrizol, Inc.) may be selected as appropriate to achieve desired lactic acid release rates.

The amount of lactic acid in the lactic acid composition may include at least one gram of lactic acid. It should be appreciated, however, that the amount of lactic acid may vary based on the therapeutic need and the capacity of the sustained-release device. For example, the amount of lactic acid in the lactic acid composition may be about 1 to about 8 grams or about 2 to about 8 grams, or about 2 grams to about 6 grams

The lactic acid composition may be in any suitable form such as a liquid, solid, semi-solid. In an embodiment, the lactic acid composition is in a substantially powder form or is in a powder form substantially free of water. In an embodiment, the powder form is at least about 95% free of water, at least about 90% free of water, or at least about 80% free of water. Powder forms of lactic acid that may be used in the disclosed sustained-release devices include, without limitation, L-lactic acid crystals such as PURAC® Powder 60 and PURAC® Powder 90, L,L-lactide, the neutral cyclic diester of lactic acid, and any other suitable powder form. In an embodiment, the amount of lactic acid to be loaded in the sustained-release device may be from about 1 to about 5 grams in the less dense powder form.

In an embodiment, the lactic acid composition is in a substantially syrup form. Syrup forms of lactic acid that may be used in the disclosed sustained-release devices include, without limitation, PURAC® PF 90 (Purac Bioquimica SA), Lactic Acid, Racemic, 85% syrup USP, (Spectrum Chemicals). In an embodiment, the amount of lactic acid to be loaded in the sustained-release device may be from about 1 to about 8 grams in syrup form.

In an embodiment, a tubular vaginal ring device made of a silicone polymer. The ring contains about one gram of a lactic acid composition in powder form within the lumen of the ring. The wall of the tubular vaginal ring device that separates the lactic acid composition from the epithelial surface of the vagina when the vaginal ring device is inserted into the vagina has a permeability that provides a ratio of lactic acid exit rate to water entry rate of at least 0.5.

In an embodiment, a tubular vaginal ring device made of a polyurethane polymer is provided. The ring contains about one gram of a lactic acid composition in powder form within the lumen of the ring. The wall of the tubular vaginal ring device that separates the lactic acid composition from the epithelial surface of the vagina when the vaginal ring device is inserted into the vagina has a permeability that provides a ratio of lactic acid exit rate to water entry rate of at least 0.5. In another embodiment the lactic acid composition within the lumen of the above device is in a syrup form.

In an embodiment, the amount of lactic acid released from a sustained-release device or composition may be at a rate of between about 1 and about 1000 milligrams of L-lactic acid per day. In an embodiment, the amount of lactic acid released from a sustained-release composition may be adapted to mimic the natural rate of vaginal L-lactic acid production by lactobacilli. Lactobacilli can produce approximately 50 million protons per second in vitro, and the human vagina contains approximately 10⁹ lactobacilli that maintain a healthy vaginal pH between 3.1 and 4.2, and a healthy vaginal lactic acid concentration between 0.8 and 1.6% (see FIG. 2). This corresponds to an estimated 650 milligrams lactic acid produced per day, with wide confidence limits due to expected differences in production rates in vivo compared to in vitro, and uncertainty regarding the rate of lactic acid leaving the vagina due to the permeability of the vaginal epithelium. Accordingly, in an embodiment, the sustained-release composition or device is adapted to release at least about 500 milligrams of lactic acid per day. In other embodiments, the sustained-release composition or device is adapted to release about 10 to about 1000 milligrams, about 400 to about 900 milligrams, or about 500 to about 800 milligrams of lactic acid per day to acidify the vagina.

It should be appreciated that the above estimated release rate requirements may be further adjusted as needed in order to achieve a desired concentration of L-lactic acid at the epithelial surface between about 0.2% and about 2%, or more preferably between about 0.5% and about 1.8% and still more preferably between about 0.8% and about 1.6%.

In an embodiment, a therapeutically effective amount of L-lactic acid is administered to a portion of the vagina. In such an embodiment, distribution of the L-lactic acid from a sustained-release composition, for example, may provide the desired concentration of L-lactic acid to a portion of the vagina near the composition. This partial distribution of the desired L-lactic acid concentration and low pH still provides an effective means to prevent and treat bacterial vaginosis, by creating a “safe harbor” for lactobacilli, which inhibit undesirable competing organisms associated with bacterial vaginosis. For this purpose, an approximately 10-fold lower release rate may be needed. Therefore, in another embodiment the amount of lactic acid released from a sustained-release composition is about 1 to about 100 milligrams of L-lactic acid per day.

It should be appreciated that there may be a relatively high concentration of L-lactic acid at the points of contact between the sustained-release composition and the vaginal epithelium. For this reason, the improved therapeutic and prophylactic index of L-lactic acid compared to D-lactic acid or racemic lactic acid is of special benefit, since the L-lactic acid will minimize potential toxicity to the epithelium at these points of contact.

In an embodiment, the sustained-release device includes a compressible gas within a lumen of the device. The compressible gas may be distributed along the lumen of the device in a compartment separate from the compartment in the lumen of the device containing the lactic acid composition. The compartment may be formed from the same material of the sustained-release device. The volume of the enclosed compressible gas may be, for example, between about 1 and about 5 mL. Air or any other inert gasses such as nitrogen gas, or combinations thereof are suitable. The amount of compressible gas in the sustained-release device is sufficient to absorb at least a portion of the increased volume of any fluid that diffuses into the lumen of the device while limiting the swelling of the device. In an embodiment, the gas may be constrained in a low compliance compartment along the inner surface of the sustained-release device. Alternatively, the gas may be provided in multiple low compliance compartments of dimensions less than the inside dimensions of the device. Such distribution of the compressible gas along the inner surface of the lumen of the sustained-release device may limit the collection of gas in a single region in the lumen and may allow lactic acid to be distributed along the inside surface of the tubing.

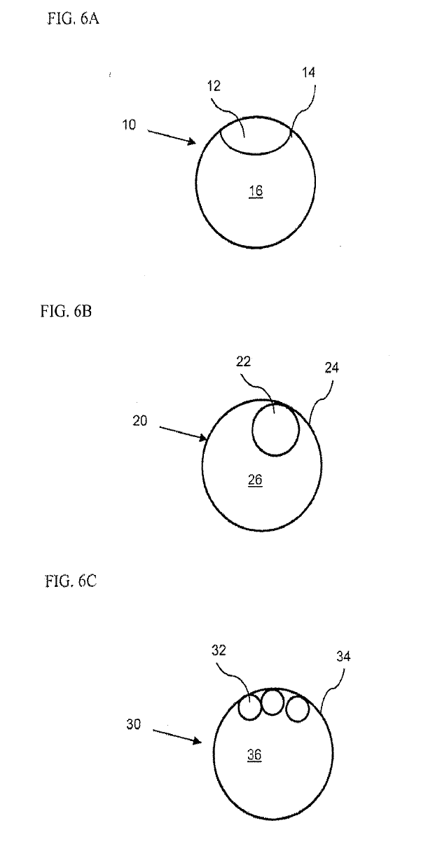

As illustrated in FIGS. 6A, 6B and 6C, a compressible gas may be provided within the device lumen. FIG. 6A illustrates a free bubble of gas 12 in the lumen 14 of the device 10, with the gas making contact with the inner wall 16, and thus limiting lactic acid contact over a portion of the inside surface 14 in contact with the gas 12, and limiting lactic acid release in this area. FIG. 6B illustrates a single cylindrical gas compartment 22 in the lumen 24 of the device 20 and making only tangential contact over a limited area of the inner wall 26, thereby increasing the area of the inside surface 24 from which lactic acid release can occur. FIG. 6C illustrates a multiple spherical gas compartments 32 in the lumen 34 of the device 10 and making tangential content with a limited area of the inner wall 36.

The sustained-release device may be compressed during loading of at least a portion of the lactic acid into the device. In an embodiment, the sustained-release device is compressed to reduce the interior volume by between about 1 and about 4 mL, or between about 10 and about 50% of the original interior volume. The lactic acid is loaded to fill the partially compressed tubing and, optionally, air is withdrawn from the lumen. After the device is loaded with the lactic acid, the internal pressure initially may be around zero but may fall to a negative pressure after the compression is released due to the elastic recoil of the tubing. This technique may enable the device to increase its capacity to absorb fluid that diffuses through the wall of the device from the genital tract surface before the device experiences positive pressure and subsequent swelling. In an embodiment, the compression-loaded device has the capacity to absorb at least several milliliters of fluid before the device experiences positive pressure and subsequent swelling beyond the original dimensions of the device before compression and loading.

It should be appreciated that one or more of the materials of the wall of the sustained-release device, the form of lactic acid, the compressible gas compartment and the compressed loading techniques described herein may be combined in an embodiment of the present disclosure.

In an embodiment, the composition is administered to a subject suffering from or at risk for suffering from a pathogenic infection of its genital tract. The pathogen may include a bacterium such as Chlamydia trachomatis, Neisseria gonorrhoeae, Gardnerella vaginalis, Porphyromonas levii, Prevotella bivia, Prevotella corporis, Anaerococcus prevotii, Fusobacterium nucleatum, Bacteroides ureolyticus, Micromonas micros, Propionibacterium acnes, Megasphaera elsdenii, Peptostreptococcus anaerobius,/Eggerthella lenta, Anaerococcus tetradius, Atopobium vaginae, Ureaplasma urealyticum, Mobiluncus curtisii, Mobiluncus mulieris, Mycoplasma hominis; a virus such as herpes simplex virus type 1 (HSV-I), herpes simplex virus type 2 (HSV-2), human papillomavirus (HPV), or human immunodeficiency virus (HIV); a yeast or fungus such as Candida albicans; a protozoan such as Trichomonas vaginalis or any combination thereof. In an embodiment, the compositions of the present disclosure are administered to overcome the alkalinizing effect of semen on the vaginal environment.

In an embodiment, a sustained-release device providing continuous release of an effective dose of lactic acid is inserted into the vagina, and retained for a period of one week, one month, or three months.

The following examples are included to illustrate the inactivation of pathogens of the genital tract by the presently disclosed compositions and are not intended to limit the scope of the present disclosure in any way.

### EXAMPLE 1

The effect of low pH alone, and low pH in the presence of racemic lactic acid, L-lactic acid, and D-lactic acid on the survival of Herpes simplex virus type 2 (HSV-2) was assessed. Cell-free HSV-2 (ATCC VR-734™ strain 9, American Type Culture Collection, Manassas, Va.) was aliquoted, stored at −80° C., and thawed immediately before its use. Elvis™ (Enzyme-linked Virus Inducible System™) cells (Diagnostic Hybrids, Athens Ohio) were supplied growing in flat-bottomed, 96-well microplates. Each plate was kept in a humidified incubator (5% CO₂, 37° C.) until the cells were between 75% and 95% confluent. The supplier's original growth medium was replaced with fresh, pre-warmed tissue culture re-feeding medium (Trinity Biotech USA, Jamestown, N.Y.) several hours before the microplate was used in an experiment.

Viral exposure media were prepared by adding 0.1% D,L-lactic acid (Sigma-Aldrich, St. Louis, Mo.), D-lactic acid (Bachem Bioscience Inc., King of Prussia, Pa.), L-lactic acid, (Sigma-Aldrich, St. Louis, Mo.), acetic acid (Sigma-Aldrich) or no acid to 0.9% NaCl in deionized water. The pH of the solutions was measured and adjusted as necessary before, during, and after the experiments using an MI-410 combination pH electrode (Microelectrodes Inc., Bedford, N.H.) to pH 3.8 for all of the organic acid-containing viral exposure media. Two viral exposure media not containing an organic acid were adjusted to pH 3.8 with HCl, or to pH 7.4 with dilute NaOH. Freshly thawed viral stock was diluted 1:10 with viral exposure media and incubated for 30 minutes at 37° C. At the end of the incubation, all media were diluted ten-fold with re-feeding medium to rapidly neutralize the acidity of those media that were acidic. Three-fold serial dilutions were then made with the same growth medium. Each dilution of virus was then inoculated into two replicate wells on a microplate of Elvis™ cells. The microplate was spun at −1000 g, 25° C. for 25 minutes and incubated in a humidified incubator (5% CO₂, 37° C.) for 16-24 hours. The growth medium was removed from the microplate wells, and the Elvis™ cells were fixed and stained according to the manufacturer's instructions. The microplate wells were examined on an inverted microscope for the presence of blue-stained cells, indicating viral infection.

### EXAMPLE 2

The effect of 30-minute exposure to 0.1% L-lactic and 0.1% D-lactic acids at pH 3.8 on the viability of Herpes simplex virus type-1 (HSV-1) was investigated. Procedures were followed as described in Example 1. The HSV-1 used was the KOS strain was (ATCC #VR-733). At pH 3.8 with 0.1% L-lactic acid, HSV-1 was inactivated approximately 80-fold more than the control at pH 7.4 without lactic acid, 25-fold more than at pH 3.8 without lactic acid. Importantly, similar to the results with HSV-2, L-lactic acid at pH 3.8 was more effective at inactivating HSV-1 than D-lactic acid at pH 3.8 (approximately 10-fold greater inactivation).

### EXAMPLE 3

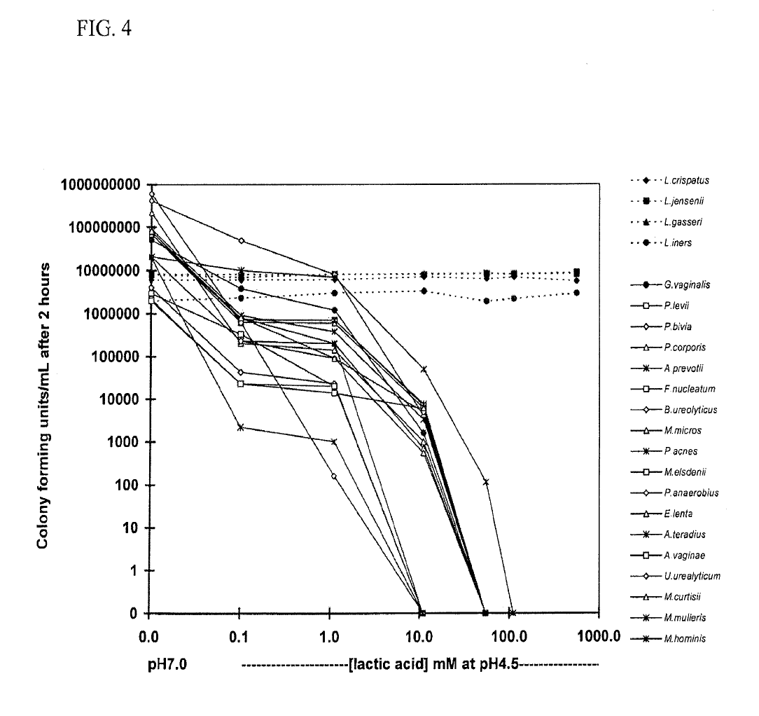

The following example shows that racemic lactic acid inactivates a wide variety of the organisms associated with the condition bacterial vaginosis (BV). Eighteen BV-associated organisms, and four lactobacillus strains obtained from the American Type Culture Collection, Rockville Md., were grown in appropriate growth medium and incubation conditions, and exposed to a 2-hour further incubation at 37° C. in media at pH 7 without lactic acid, or at pH 4.5 with racemic lactic acid concentrations ranging from zero to 550 mM (5%). The number of organisms surviving exposure to these conditions is plotted in FIG. 4. BV-associated organisms are plotted with solid lines. The maximum viability is seen on the far left of the figure, at pH 7, and with no lactic acid present. All BV-associated organisms showed a substantial drop in viability at pH 4.5 without lactic acid, shown plotted one position to the right. Inactivation was dramatically increased at pH 4.5 in the presence of between 10 and 100 mM (˜0.1-1%) lactic acid. In contrast, lactobacilli survived unaltered at pH 4.5 even at the highest concentration of lactic acid tested (5% or ˜550 millimolar). These data demonstrate that lactic acid is a potent inhibitor of BV-associated organisms, and provides substantially more inactivation than low pH alone. Moreover, this activity is advantageously directed against BV-associated organisms, while sparing lactobacilli, plotted with dotted lines, the normal and protective flora of the human vagina. Since 550 mM lactic acid (0.5%) had no effect on lactobacilli, and since racemic lactic acid is 50% L-lactic acid, these data also indicate that concentrations up to 280 mM (0.25%) L-lactic acid is not harmful to lactobacilli.

### EXAMPLE 4

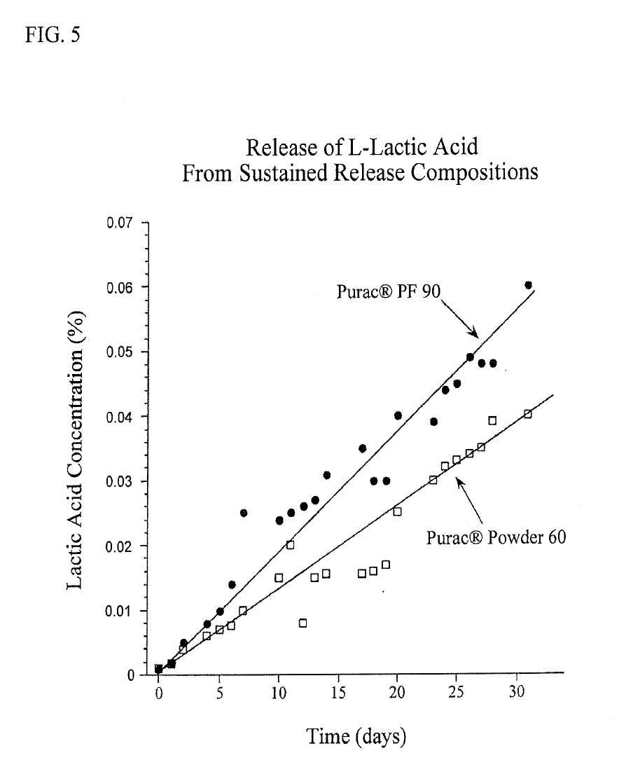

Three of the BV-associated organisms studied in Example 3, two Mobiluncus species (M. mulieris, and M. curtisii) and a third, phylogenetically distinct (i.e., from another bacterial division) bacterium, Mycoplasma hominus, were studied with the methods described above, but in this case exposed to L-lactic acid rather than racemic lactic acid. As with racemic lactic acid, inactivation of all three organisms was substantially greater at pH 4.5 with L-lactic acid than at pH 4.5 without lactic acid. These results demonstrate the effectiveness of L-lactic acid in a variety of BV-associated organisms listed in FIG. 5. It should be appreciated, therefore, that the effectiveness of L-lactic acid is sufficiently broad to be expected to inactivate other BV-associated organisms and, thus, provide effective prevention and therapy against bacterial vaginosis.

### EXAMPLE 5

The effect of L-lactic acid on the viability of HIV-1 is investigated by adding high titer HIV viral stock (strain IIIB) to three different exposure media: PMI medium with 5% serum containing 0.5% L-lactic acid, RPMI medium with 5% serum without any lactic acid and adjusted to pH 4.5, and RPMI medium with 5% serum without lactic acid and adjusted to pH 7.4. Viral stock is added to each exposure medium, while the medium is held at 37° C., constantly stirred, and constantly monitored with an MI-410 combination pH electrode (Microelectrodes Inc., Bedford, N.H.). If required, pH is adjusted back to the original pH of the exposure medium immediately after addition of virus. The mixture is incubated 30 min, then diluted 1:10 with RPMI medium with 5% serum and 25 mM HEPES buffer to restore the pH to 7.4, and assayed on susceptible cells by endpoint dilution. Viability is assessed by titering on an indicator cell line that expresses an enzyme induced by HIV infection, exposing the tissue culture cell monolayers to a substrate that is converted to a colored precipitate in infected cells, and counting the cells with visible colored precipitate. Viability of HIV-1 is expected to be modestly reduced by incubation at pH 4.5 without L-lactic acid, compared to the control incubation. Like HSV-1 and HSV-2, HIV is also an enveloped virus; thus, the viability of HIV-1 is expected to be reduced to a substantially greater degree by incubation at pH 4.5 in medium containing 0.5% L-lactic acid compared to pH 4.5 without lactic acid.

### EXAMPLE 6

The physiologic levels of natural protectants in healthy individuals serve as important guides to safe levels to be used in preventive or therapeutic compositions. Prior measurements of pH and organic acid concentrations of human vaginal secretions have been compromised by measurement artifacts caused by observations under aerobic conditions rather than the anaerobic conditions that are physiological to the vaginal lumen. Thus, these former methods underestimated the lactic acid concentration, since, at the elevated oxygen concentration of ambient air, lactic acid is rapidly metabolized to acetic acid. Likewise, these former methods overestimated the pH value, since carbon dioxide is quickly lost from secretions once exposed to ambient air leading to substantial pH shifts, and since the pKa of acetic acid is higher than that of lactic acid. For this reason, measurements were made under physiologic (anaerobic) conditions to guide the choice of appropriate pH and lactic acid concentrations to use in vaginal compositions for inactivation of pathogens. Freshly obtained secretions from women with lactobacillus-dominated vaginal flora assessed by Gram stain were obtained by a published vaginal fluid sampling method using the Instead® menstrual cup (Instead, Inc., San Diego, Calif.), and handled in anaerobic nitrogen atmosphere in a glove box. pH was measured with an MI-410 combination electrode within 1 minute of obtaining the secretions. Lactic acid was determined with an enzymatic method (D-Lactic acid/L-Lactic acid Enzymatic BioAnalysis/Food Analysis UV method (R-Biopharm, Darmstadt, Germany), again conducted in an anaerobic environment (nitrogen filled glove box). Measurements of pH and lactic acid concentration were made in freshly obtained cervicovaginal secretions under physiologic (i.e., anaerobic) conditions to determine the appropriate levels of pH and lactic acid concentration for preventive and therapeutic compositions based on acid buffers supplemented with L-lactic acid. FIG. 2 illustrates a plot of pH vs. total lactic acid concentration. Mean vaginal pH is 3.7 (range 3.1-4.2), and mean total lactic acid concentration is 1.2% (w/v) (range 0.8-1.6%).

These data were used to guide the choice of lactic acid concentration and pH of a composition appropriate for vaginal use in the following exemplary Composition A: five grams of L-lactic acid (Sigma Chemical Company) was added to 935 grams of USP water and mixed to homogeneity. To this solution 38 grams of carbomer (Carbopol® 974P NF (Lubrizol corporation)) was dispersed by gradual addition during vigorous mixing with a high sheer Lightnin® mixer equipped with a 3 inch diameter propeller. A countermotion mixer was substituted for the propeller mixer and the mixing was continued during addition of 10 N sodium hydroxide added until the final pH was pH 3.9, and USP water q.s, to 1000 grams total weight.

Other exemplary compositions were prepared in accordance with the present description with the following variations in formula:

Composition B: USP deionized water, 1% carboxymethylcellulose, 4% Carbopol® 974P, 0.5% L-lactic acid, and potassium hydroxide quantity sufficient (q.s.) to adjust the pH to 3.8;

Composition C: USP deionized water, 1% carboxymethylcellulose, 4% Carbopol® 974P, 0.5% L-lactic acid, 0.3% monobasic sodium phosphate, and dibasic potassium phosphate q.s. to pH 3.8;

Composition D: USP deionized water, 2% alginic acid, 3.5% Carbopol® 974P, 0.5% L-lactic acid, and potassium hydroxide q.s. to pH 3.8;

Composition E: USP deionized water, 1% carboxymethylcellulose, 4% Carbopol® 974P, 0.5% L-lactic acid, 0.1% sorbic acid, and potassium hydroxide q.s. to pH 3.8; and

Composition F: USP deionized water, 1% carboxymethylcellulose, 4% Carbopol 974P, 0.5% L-lactic acid, 1% K₂HPO₄, 0.2% NaH₂PO₄, sodium hydroxide q.s. to pH 3.8.

### EXAMPLE 7

The toxicity of D-lactic acid and of L-lactic acid were assessed on tissue culture monolayers using a well known cell viability assay. HeLa cells (ATCC CCL-2, a cell line derived from a cervical epithelial cancer) were grown in 96-well tissue culture plates to approximately 90% confluence in DMEM medium supplemented with 3% fetal bovine serum. Medium was removed from the wells, and replaced by the same medium now containing D- or L-lactic acid between concentrations of 0% and 1% (w/v). The plates were incubated for 30 minutes at 37° C. and 5% CO₂. The wells were washed three times with 0.9% saline, and then 20 microliters of a tetrazolium viability stain (Promega CellTiter 9603 One Solution) was added. After 1 hour, the absorbance of the fluid in the wells was read with a spectrophotometer at a wavelength of 575 nanometers.

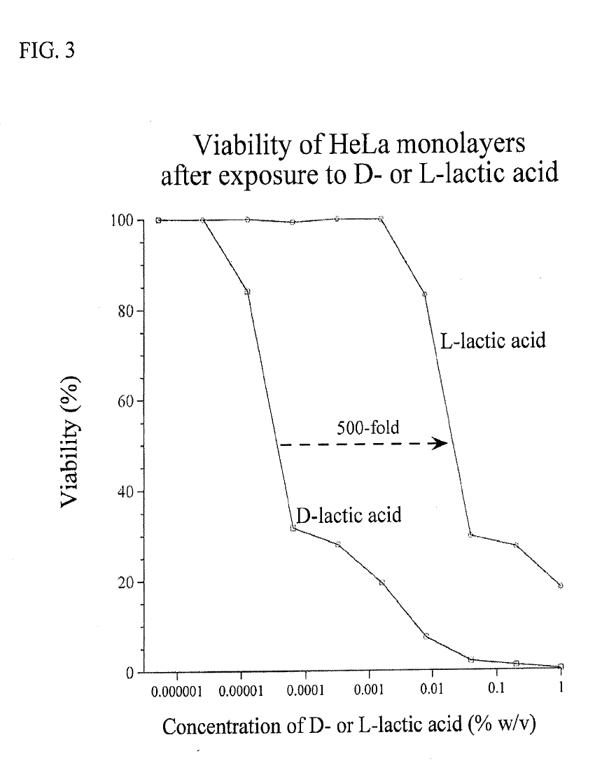

Results of this assay are plotted in FIG. 3, as percent viability (the ratio of the optical density of the lactic acid treated wells to the optical density of the medium-only treated wells). It is evident from the illustrated results that the dose-response for cellular cytotoxicity of L-lactic acid is shifted more than 500-fold to the right, that is, it took more than 500-fold more L-lactic acid to have the same degree of cytotoxicity as was seen with D-lactic acid.

### EXAMPLE 8

The efficacy of each of the exemplary compositions in Example 6 is assessed as in Examples 1, 2 and 3. Based on the data and findings from Examples 1, 2 and 3, each of the compositions is expected to inactivate pathogens to a greater degree than compositions containing D-lactic acid or D,L-lactic acid. The toxicity of each of the exemplary compositions in Example 4 is assessed according to the procedures described in Example 8. Based on the data of Example 7, each of the compositions is expected to demonstrate reduced toxicity to epithelial cells compared to D-lactic acid and D, L-lactic acid.

### EXAMPLE 9

In light of the greater cytotoxicity of D-lactic acid compared to L-lactic acid in epithelial cell monolayers described above, the in vivo toxicity of the D- and L-stereoisomers of lactic acid was assessed in an animal model that measures changes in vaginal susceptibility to an important sexually transmitted pathogen, Herpes simplex type 2 (HSV-2). In this model, the composition to be tested is applied 12 hours before vaginal challenge with the viral pathogen. At this time (12 hours) after exposure to the composition, the protective effect of the composition has dissipated, and it is possible to assess for harmful effects of the exposure that may result in increased susceptibility to infection. The proportion of animals infected after prior exposure to the composition was compared to the proportion infected after exposure to a control agent. A difference in the proportion of animals infected reveals toxicities of the prior application of the composition that increases susceptibility to infection.

Three compositions were prepared for testing in this in vivo model: an acid buffering gel with 3.8% Carbopol® 974P NF with 0.5% L-lactic acid; an acid buffering gel with 3.8% Carbopol® 974P NF with 0.5% D-lactic acid; and an acid buffering gel with 3.8% carbomer with 1% D,L-lactic acid (racemic lactic acid). All were prepared as described in Example 6 and with formulation pH of 3.9.

Female CF-1 mice 6-8 weeks old (Harlan, Indianapolis, Ind.) were acclimatized for 1-2 weeks after shipping, then injected subcutaneously with 2.5 mg Depo-Provera® (medroxyprogesterone acetate) (Pharmacia & Upjohn Company, Kalamazoo, Mich.). Twenty microliters of the test composition was delivered to the vagina, and 12 hours later a low-dose inoculum with 0.4 ID₅₀ was delivered in 10 microliters of Bartels Tissue Culture Refeeding Medium (Trinity Biotech, St. Louis, Mo.). Strain G of HSV-2 (ATCC lot #3405329) was obtained from Virotech International (Rockville, Md.; 5×10⁸ tissue-culture-infectious-dose-50% (TCID₅₀)/ml). The viral stock was thawed and refrozen in 100 microliter aliquots, then stored at −70° C. A thawed aliquot of viral stock was diluted with Bartels Medium (Trinity Biotech, St. Louis, Mo.) to yield an inoculum with 10 ID₅₀ in a 10 microliters inoculum (˜10⁴ TCID₅₀). The viral stock was further diluted with Bartels Medium as needed. The diluted viral stock was stored on ice and used within one hour of thawing. The 10 microliters viral inoculum was delivered with a Wiretrol pipette (Drummond Scientific, Broomall, Pa.) with a fire-polished tip to minimize potential injury. Vaginal lavages were obtained 3 days after inoculation and evaluated for viral shedding. Fifty microliters of Bartels Medium was delivered to the vagina and pipetted in and out 20 times to maximize viral recovery, then diluted into 50 microliters Bartels Medium in a 0.5 ml microfuge tube. The vaginal lavage samples were then spun at 6500 rpm in a microcentrifuge for 5 minutes to pellet the cells and mucus. The pellet was then removed using a pipette tip to draw the pellet up the side of the tube and out of the supernatant. The supernatant was then placed on target cells (human newborn foreskin diploid fibroblast cells; Biowhitaker, Walkersville, Md.). Cytopathic effect was scored 48 hours later, and mice whose lavage cultures displayed cytopathic effect were considered infected.

The first experiment (reported in Table 1) compared delivery of the test composition with 0.5% L-lactic acid in 3.8% carbomer (Carbopol® 974P NF), injection of the test composition with 0.5% D-lactic acid in 3.8% carbomer, and sham delivery (instrumentation of the vagina with the Wiretrol pipette, but without delivery of any composition).

TABLE 1

| | | | | Percent |
|---|---|---|---|---|
| Pretreatment | Infected | Uninfected | Total | Infected |
| Gel with 0.5% L-Lactic Acid | 14 | 26 | 40 | 35% |
| Gel with 0.5% D-Lactic Acid | 29 | 11 | 40 | 72.5% |
| Sham inoculation | 18 | 22 | 40 | 45% |

The data in Table 1 show that delivery of 0.5% L-lactic acid in a carbomer-based acidifying gel did not increase susceptibility to HSV-2 vaginal infection compared to sham delivery. In contrast, delivery of an acidifying gel containing 0.5% D-lactic acid caused toxicity manifest as a significantly increased susceptibility to HSV-2 vaginal infection both when compared to sham delivery (P<0.025), and when compared to delivery of the acidifying gel with 0.5% L-lactic acid (P<0.002).

The second experiment (reported in Table 2) compared injection of the test composition containing 1% D,L-lactic acid in a 3.8% carbomer acidifying gel with sham injection.

TABLE 2

| | | | | Percent |
|---|---|---|---|---|
| Pretreatment | Infected | Uninfected | Total | Infected |
| Gel with 1% D,L-Lactic Acid | 22 | 8 | 30 | 73% |
| Sham inoculation | 12 | 18 | 30 | 40% |

The data in Table 2 show that animals pretreated with the acidifying gel containing 1% D,L-lactic acid showed significantly more infections than sham inoculated animals (P=0.002).

Thus, the data in Tables 1 and 2 demonstrate that L-lactic acid substantially free of D-lactic acid is advantageous as a component of an acidifying vaginal gel since it is less toxic than compositions containing D-lactic acid, or racemic lactic acid. Moreover, the data in Tables 1 and 2 demonstrate the suitability of L-lactic acid for inclusion in a prophylactic microbicide preparation. In contrast, preparations containing D-lactic acid, either alone, or as part of a racemic mixture of D,L-lactic acid tend to increase risk of infection due to toxicity which is intolerable in a preparation that must reduce transmission of infection to fulfill its intended purpose.

### EXAMPLE 10

The following example demonstrates release of L-lactic acid over an extended period of time from a composition suitable for placement in the vagina. The composition was assembled from silicone tubing ( 5/16th inch OD, 3/16th inch ID, wall 1/16th inch, platinum-cured MedX silicone tubing, Small Parts, Inc., Miramar, Fla.) formed into a ring by inserting a barbed polypropylene tubing connector into the two free ends of the tubing. Before sealing the tubing was loaded with 3.4 mL PURAC® PF 90 (pyrogen free L(+)-lactic acid, 90% concentration, less than 1% D-lactic acid stereoisomer, Purac Bioquimica SA, Barcelona, Spain), This fluid had a density of 1.2 grams/milliliter, and thus the volume loaded contained approximately 3.6 grams of L-lactic acid. The composition was immersed in 300 mL of pH 4.0 citrate buffer, containing gentamicin and amphotericin to prevent microbial growth, and incubated at 37° C. without stirring except briefly immediately before daily withdrawal of samples for analysis of L-lactic acid with an enzymatic assay (D-Lactic acid/L-lactic acid Enzymatic BioAnalysis/Food Analysis UV method, (R-Biopharm, Darmstadt, Germany). A second composition was constructed and evaluated in the same fashion except for substitution of PURAC® powder 60: (60% L-lactic acid, 37% calcium lactate, Purac Bioquimica SA, Barcelona, Spain) for the L-lactic acid liquid in the first composition. FIG. 5 shows the concentration accumulating in the incubation buffer over the course of the experiment for each sustained-release composition.

These data show that L-lactic acid can be released across a permeable membrane at a steady rate over a prolonged period, as would be advantageous to provide long-term protection against acquisition of a new infection from an outside source, and also to protect against the increase of harmful endogenous flora such as the organisms associated with bacterial vaginosis. It further demonstrates that different physical forms of lactic acid (e.g. solid, liquid, fully acidic, partially neutralized, etc.) can be incorporated into such compositions, and successfully released at a steady rate. It should be appreciated that multiple parameters can be altered to increase or decrease the release rate in order to achieve the desired concentration of L-lactic acid (0.2-2.0%) and pH (3.1-4.2) at the epithelial surface of the vagina, including, varying the overall and cross sectional diameter of the ring, the wall thickness of the tubing, the concentration and pH of the enclosed L-lactic acid.

### EXAMPLE 11

The following example describes a sustained-release composition and means of assembling it. The composition is assembled from a 9 inch long segment of silicone tubing ( 3/16th inch ID, wall 1/32^{nd} inch, platinum-cured MedX silicone tubing. A ½ inch long silicone solid cylinder of 3/16^{th} inch OD is cemented in one end to a depth of ¼^{th} inch with silicone adhesive. After curing, the tubing is positioned with the remaining open end upward, and filled to ½ inch of the top with PURAC® PF 90 (90% w/v L-lactic acid, pH 0.5, less than 1% D-lactic acid sterioisomer). A ⅛^{th} inch long needle-vented silicone plug of 3/16^{th} inch OD is inserted to the level of the L-lactic acid column, and the needle removed. The lower end of the tubing is brought around to form a ring, and the projecting silicone cylinder coated with silicone adhesive, and inserted into the space above the ⅛^{th} inch long plug.

### EXAMPLE 12

The following example describes an additional sustained-release composition and means of assembling it. The composition and assembly are identical to that described in Example 11, except the lactic acid is PURAC® powder 60: (60% L-Lactic acid, 37% calcium lactate, pH 3.5).

### EXAMPLE 13

Four types of rings were prepared using connectors as described in Example 10, and with tubing and L-lactic acid as described in Table 3. The silicone tubing was as described in Example 10, and the polyurethane rings were made from polyether polyurethane. The vaginal rings included an outer diameter of 5/16th inch, an inner diameter of 0.25 inch, a wall thickness of 0.032 inch, and the length of the tubing outside the connector was 19 cm. The liquid L-lactic acid was PURAC® PF 90 as described in Example 10, and the powder was PURAC® Powder 60 from the same source. Three rings of each type were immersed in 40 mM citrate buffer, pH 4.0, with gentamicin and amphotericin microbial growth inhibitors. At the end of 31 days incubation, the weight and cross-sectional diameter of the rings were determined. The degree of pressurization of the rings was determined by determining the force in newtons required to compress each ring by 3 mm with a chisel tip and Extech 475040 Force Gauge. The rings were disassembled, and the residual L-lactic acid inside the ring was measured by the enzymatic assay. The empty rings were reassembled, and the compression measurement repeated and subtracted from the pressurization measurement to calculate the net pressurization due to the ring contents. The water uptake by the rings was determined by adding the weight gain of the ring during the buffer incubation to the weight of L-lactic acid released during the incubation, which was in turn calculated b subtracting the initial weight of L-lactic acid from the residual L-lactic acid content after the incubation. All values reported in Table 3 are the mean values of the measurements for three replicate rings of each type.

TABLE 3

| Ring type | A | B | C | D |
|---|---|---|---|---|
| Tubing material | Silicone | Polyurethane | Silicone | Polyurethane |
| Form of L-Lactic acid (LA) | LA syrup | LA syrup | LA powder | LA powder |
| LA loaded (grams) | 4.16 | 6.87 | 2.08 | 3.90 |
| LA remaining (grams) | 2.25 | 1.36 | 2.035 | 1.91 |
| LA released/31 days (grams) | 1.74 | 5.022 | 0.041 | 1.82 |
| LA average release/day (grams) | 0.056 | 0.162 | 0.0013 | 0.059 |
| Water uptake (grams) | 4.01 | 5.01 | 1.17 | 2.83 |
| Ratio of LA release to water uptake | 0.43 | 1.00 | 0.03 | 0.64 |
| Pressurization (newtons) | 2.74 | 0.34 | 0 | 0.11 |
| Increase in ring cross-sectional | 1.2 | 0.3 | 0.2 | 0.1 |
| diameter (mm) | | | | |

In the Type A rings, composed of silicone tubing and filled with about 4 grams of the standard pharmaceutical L-lactic acid preparation (USP grade L-lactic acid syrup), the high osmolality inherent in the high concentration and high total mass of L-lactic acid resulted in substantial water uptake, pressurization, and swelling of the ring. This result also indicates that silicone has a higher permeability for water. Such a degree of pressurization is disadvantageous since it progressively alters the compressibility of the ring, and may change its ease of insertion and removal. Furthermore, pressurization of the L-lactic acid contents may create a risk of leakage of the potentially caustic high concentrations of L-lactic acid through any possible imperfection or deterioration of the sealing of the ring. Without being bound to any hypothesis or theory, pressurization and swelling in Type A rings occurred because the rate of L-lactic acid exiting was less than half the rate of water entering, and because the L-lactic acid syrup filling the ring is an incompressible fluid.

In the Type B rings, polyurethane was substituted for silicone as the ring material and diffusive barrier to water and L-lactic acid. The wall thickness was half that of the silicone rings, resulting in a higher capacity for loading with L-lactic acid syrup. L-lactic acid release was 2.9-fold higher than that seen with Type A rings, higher than the expected 2-fold higher that would have resulted solely from the 2-fold thinner wall, and indicative of an increased permeability to L-lactic acid of the polyurethane material relative to the silicone material. Minimal pressurization and minimal change in crosssectional diameter of the ring was observed. Although the rate of L-lactic acid exit closely matched the rate of water entry through the polyurethane tubing wall, the ratio of permeability to L-lactic acid relative to water appears to be higher than this ratio through silicone.

In the Type C rings, silicone tubing material was combined with L-lactic acid loaded in the form of a partially neutralized powder (PURAC® Powder 60). Reduced water uptake, minimal swelling, and lack of pressurization, were advantageously observed with Type C rings. In addition, the combination of silicon ring material and L-lactic acid powder resulted in a reduced rate of 1.3 mg/day L-lactic acid release.

The Type D rings were constructed of polyurethane and filled with the PURAC® Powder 60 form of L-lactic acid. These rings, like the Type B and Type C rings showed minimal swelling or pressurization due to a fairly high ratio of L-lactic acid release to water entry rate.

Although a high loaded mass of L-lactic acid is an important factor in creating the potential problem of ring swelling and pressurization, water uptake and pressurization did not correlate with mass of L-lactic acid loaded into Type A-D rings.

### EXAMPLE 14

A polyurethane vaginal ring having a wall thickness of about 1 mm is loaded with about 5 grams of L-lactic acid to provide continuous release of approximately 100 milligrams L-lactic acid per day into the vagina over about one month for the prevention of bacterial vaginosis.

It should be understood that various changes and modifications to the presently preferred embodiments described herein will be apparent to those skilled in the art. Such changes and modifications can be made without departing from the spirit and scope of the present invention, and without diminishing its intended advantages. It is therefore intended that such changes and modifications be covered by the appended claims.

1. A device comprising at least one wall separating a composition from a genital tract surface, said composition comprising at least one gram of lactic acid, wherein the permeability of said wall for said lactic acid is at least half the permeability of said wall for water.

2. The device of claim 1, wherein the wall is in the form of a tube.

3. The device of claim 2, wherein the tube is in the shape of a ring.

4. The device of claim 3, wherein the tubular ring includes an outer diameter of between about 50 mm and 100 mm and a cross-sectional diameter between about 4 mm and about 15 mm.

5. The device of claim 1, wherein the wall includes a silicone polymer.

6. The device of claim 5, wherein the lactic acid is in a powder form.

7. The device of claim 1, wherein the wall includes a polyurethane polymer.

8. The device of claim 7, wherein the lactic acid is in a liquid or powder form.

9. The device of claim 1, wherein the composition includes at least one form of lactic acid selected from the group consisting of L-lactic acid, calcium lactate and L,L-lactide.

10. The device of claim 1, wherein the composition includes between about 1 gram and about 8 grams of lactic acid.

11. The device of claim 1, wherein the lactic acid passes through the wall at a rate of about 1 milligram to about 1000 milligrams per day.

12. A method of decreasing the pH of a genital tract comprising positioning within the genital tract for at least one week a device comprising at least one wall separating a composition from the genital tract surface, said composition comprising at least one gram of lactic acid, wherein the permeability of said wall for said lactic acid is at least half the permeability of said wall for water.

13. The method of claim 12, wherein the pH at the epithelial surface of the genital tract is decreased to between about 3.1 and about 4.2.

14. A method of treating a pathogenic infection of a genital tract comprising positioning for at least one week within the genital tract a device comprising at least one wall separating a composition from the genital tract surface, said composition comprising at least one gram of lactic acid, wherein the permeability of said wall for said lactic acid is at least half the permeability of said wall for water, and wherein the pathogenic infection is selected from the group consisting of bacterial vaginosis, Chlamydia trachomatis, Neisseria gonorrhoeae, herpes simplex virus type 1, herpes simplex virus type 2, human immunodeficiency virus (HIV), Trichomonas vaginalis, and combinations thereof.

15. A device for delivering a high dose of lactic acid to a genital tract comprising:
lactic acid in a substantially powder form;
a wall separating the lactic acid from a surface of the genital tract, said wall including a polyurethane polymer, wherein the permeability of said wall for said lactic acid is at least half the permeability of said wall for water; and
a compartment including a gas, wherein the compartment extends along the length of the device.

16. The device of claim 15, wherein compression is applied to the device during loading said device with the lactic acid, and wherein said compression is released after loading said device with said lactic acid.

17. A genital tract surface device comprising:
a tubular ring having a wall, said wall defining a space within said tubular ring, wherein said space contains at least one gram of an L-lactic acid composition, substantially free of D-lactic acid, and wherein the rate of diffusion of L-lactic acid from the space through the wall of said tubular ring is at least half the rate of diffusion of water through the wall into the space.

18. The device of claim 17, wherein the wall includes a polyurethane polymer.

19. The device of claim 17, wherein the lactic acid is in a substantially powder form.

20. (canceled)

21. The method of claim 14 comprising preventing infection of the vaginal epithelium.
